(19) United States
(12) Patent Application Publication
(10) Pub. No.: US 2025/0025136 A1
(43) Pub. Date: Jan. 23, 2025

(54) DEVICE AND METHOD FOR SPLIT SALIVA COLLECTION AND TESTING

(71) Applicant: Premier Biotech, LLC, Minneapolis, MN (US)

(72) Inventors: Larry R. MASTERSON, Minneapolis, MN (US); Todd BAILEY, Bonita Springs, FL (US)

(21) Appl. No.: 18/775,773

(22) Filed: Jul. 17, 2024

Related U.S. Application Data
(60) Provisional application No. 63/527,249, filed on Jul. 17, 2023.

(51) Int. Cl. A61B 10/00

(52) U.S. Cl. CPC A61B 10/0051; A61B 2010/0006

(57) ABSTRACT

A bodily fluid sample device comprises a wand body having an elongate configuration. The wand body has a first body portion and a second body portion, the wand body being initially unitary but configured so that the first body portion and second body portion can be separated from one another. For example, the wand body may define a line of perforations at which the first body portion and the second body portion can be separated from each other. A first collection pad is located on the first body portion and a second collection pad is located on the second body portion.

### CROSS-REFERENCE TO A RELATED APPLICATION

This application is based upon and claims priority to provisional application Ser. No. 63/527,249, filed Jul. 17, 2023, which is incorporated fully herein by reference for all purposes.

### FIELD OF THE INVENTION

The present invention relates to devices and methods for collecting and testing saliva. Embodiments of the present invention relate to saliva collection and stabilization of the sample prior to testing for one or more substances.

### BACKGROUND OF THE INVENTION

Bodily fluids are collected for a variety of applications that may include, but are not limited to, diagnosis for illness, determining the presence of illicit substances, confirming or establishing levels of therapeutic agents, and RNA/DNA profiling. For such purposes, the types of bodily fluids collected include blood, urine, hair, and saliva. Saliva often represents compounds that are currently in the system, similar to blood, but the collection of saliva is minimally invasive as compared to blood collection. Privacy concerns make direct observation of saliva collection more facile as opposed to urine collection. This makes saliva advantageous in situations such as drugs of abuse testing and RNA/DNA testing. Last, the analysis of hair is typically associated with the presence of compounds that were present in an individual weeks or months prior, which limits the utility in the detection of illicit substances that may be life threatening or for situations such as impaired driving under the influence of drugs.

Split specimens are often used in laboratory testing where one sample is used for testing at one location or time, while the other is preserved for future analysis or used for testing at another location or time to confirm reproducibility or for retesting needs. Split specimens should represent identical samples. Identical samples are the same sample type (blood, urine, hair, or saliva) taken at the same time from an individual. Split specimens for urine and blood can be quickly accommodated by directly dividing a large aliquot of sample collected. For urine and blood, this is easily done since these specimens possess fluidity that resembles water and can be split. Two specimens may also be collected for blood by simply changing the vessel used during collection. Further, the volume of these specimens is often tens of milliliters (mL), which makes handling of the sample at the point of collection much easier when the sample needs to be divided. So long as sufficient hair specimen is available, dividing hair specimens can be done by mass after collection or by taking two samples near the same time. Saliva poses very different challenges when it comes to split sample collection. This is due to the lower volume of collection (between 1-3 mL) and the inherent viscosity of saliva which makes handling it much more difficult at the point of collection.

The present invention recognizes and addresses considerations of prior art constructions and methods.

### SUMMARY OF THE INVENTION

Embodiments of the present invention provide a saliva collection device and buffer system that is intended to provide multiple identical saliva samples for laboratory testing. The collection wand preferably includes two (or more) separate and equal sized collection pads that are placed next to each other. The wand has an observation window for each collection pad, which ensures that each pad has collected sufficient volume. The wand is also perforated in the middle so that it can be easily separated into two halves. After collection is complete, each half can then be inserted into a buffer bottle for storage prior to laboratory testing. The resulting process provides at least two identical saliva samples that were acquired at the same time of collection.

According to one aspect, the present invention provides a bodily fluid sample device comprising a wand body having an elongate configuration. The wand body has a first body portion and a second body portion, the wand body being initially unitary but configured so that the first body portion and second body portion can be separated from one another. For example, the wand body may define a line of perforations at which the first body portion and the second body portion can be separated from each other. A first collection pad is located on the first body portion and a second collection pad is located on the second body portion.

According to some exemplary embodiments, the first collection pad and the second collection pad are adjacent to one another near a first end of the wand body.

According to some exemplary embodiments, each of the first body portion and the second body portion has a respective first wicking element and a second wicking element in fluid communication with the respective first collection pad and the second collection pad, the wicking elements providing a visual indication that a sample is present.

According to some exemplary embodiments, the first wicking element and the second wicking element may each have an elongate configuration.

According to some exemplary embodiments, the first wicking element and the second wicking element may comprise paper having dye that changes color when the sample is present.

According to some exemplary embodiments, each of the first and second wicking elements may extend under a respective first and second indicator window, the indicator windows being located near a second end of the wand body opposite the first end.

According to some exemplary embodiments, the wand body may have a multilayer configuration with the first and second wicking elements being sandwiched between two layers.

Another aspect of the present invention provides a bodily fluid sample device comprising a wand body having an elongate configuration. The wand body has a first body portion and a second body portion, the wand body being initially unitary but having a separation line so that the first body portion and second body portion can be separated from one another. A first collection pad is located on the first body portion and a second collection pad is located on the second body portion, the first collection pad and the second collection pad being adjacent to one another near a first end of the wand body. Each of the first body portion and the second body portion has a respective first wicking element and a second wicking element in fluid communication with the respective first collection pad and the second collection pad, the wicking elements providing a visual indication that a sample is present at a respective first and second indicator window.

A still further aspect of the present invention provides a method of collecting a saliva sample from a user. One step of the method involves providing a bodily fluid collection device having an elongate wand body with a first body portion and a second body portion having respective collection pads, the wand body being configured so that the first body portion and second body portion can be separated from one another. According to another step, a sample is collected at the collection pads. According to another step, it is determined if the sample is sufficient. If the sample is sufficient, the first body portion is separated from the second body portion. A further step involves separately storing the first body portion and the second body portion.

The accompanying drawings, which are incorporated in and constitute a part of this specification, illustrate one or more embodiments of the disclosure and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended drawings, in which:

FIG. 1 is a top perspective view of a saliva collection device in accordance with an embodiment of the present invention;

FIG. 2 is a bottom perspective view of the device of FIG. 1;

FIG. 3 is a top plan view of one-half of the device of FIG. 1;

FIG. 4 is a cross-section view taken along line 4-4 of FIG. 3;

FIG. 5 is a top perspective view of one-half of the device of FIG. 1 with a top layer removed to reveal a wicking element;

FIG. 6 shows separation of two portions (two halves) of the device after a sufficient quantity of specimen has been collected; and

FIG. 7 is a flow chart illustrating steps according to methodology of the present invention.

Repeat use of reference characters in the present specification and drawings is intended to represent same or analogous features or elements of the invention according to the disclosure.

### DETAILED DESCRIPTION OF PREFERRED EMBODIMENTS

Reference will now be made in detail to presently preferred embodiments and presently preferred methodology of the invention, one or more examples of which are illustrated in the accompanying drawings. Each example is provided by way of explanation, not limitation, of the invention. In fact, it will be apparent to those skilled in the art that modifications and variations can be made in the present invention without departing from the scope and spirit thereof. For instance, features illustrated or described as part of one embodiment (or method) may be used on another embodiment (or method) to yield a still further embodiment (or method). Thus, it is intended that the present invention covers such modifications and variations as come within the scope of the appended claims and their equivalents.

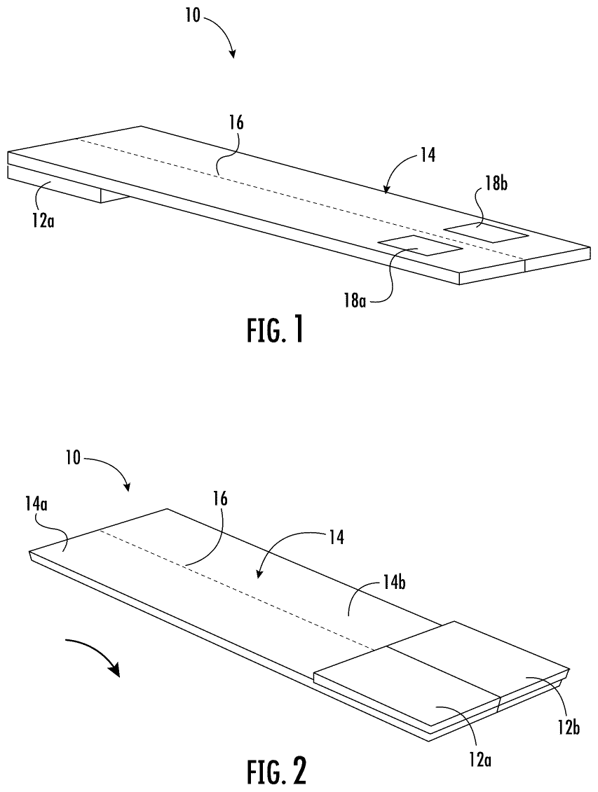

As used herein, terms referring to a direction or a position of the sample device, such as but not limited to “vertical,” “horizontal,” “top,” “bottom,” “above,” or “below,” refer to directions and relative positions with respect to the device's orientation shown in FIG. 1. Further, the term “or” as used in this document is intended to mean an inclusive “or” rather than an exclusive “or.” That is, unless specified otherwise, or clear from the context, the phrase “X employs A or B” is intended to mean any of the natural inclusive permutations. Therefore, the phrase “X employs A or B” is satisfied by any of the following instances: X employs A; X employs B; or X employs both A and B. In addition, the articles “a,” “an,” and “the” as used in this document should generally be construed to mean “one or more” unless specified otherwise or clear from the context to be directed solely to a singular form. The meaning of “in” may include “in” and “on.” The word “at” may include “at,” “adjacent to,” and “on.” The phrase “in one embodiment,” as used herein does not necessarily refer to the same embodiment, although it may. The meanings identified above do not necessarily limit the terms, but merely provide illustrative examples for the terms.

Aspects of the present invention provide a solution for the collection and subsequent splitting of a saliva sample utilizing a novel collection wand. For example, the collection wand may have two separate and equal sized collection pads that are placed next to each other. The wand may also have separate collection windows that provide the user with a visual indication of sufficient volume collected on each separate pad. During collection, the wand remains intact as a single piece. The wand is perforated or otherwise weakened in the middle so that it can be easily separated into two halves. This can be done after collection is completed and before the sample is split to represent two samples. Two containers containing storage buffer may be used to preserve the analytes of interest during transport and storage.

FIGS. 1 and 2 illustrate a bodily fluid collection device constructed in accordance with an embodiment of the present invention, here in the form of a collection wand 10. In this case, wand 10 preferably includes a plurality of collection pads for collecting respective essentially identical samples. In this case, two separate and equal sized collection pads 12a and 12b are placed next to each other, as shown. Specifically, the pads 12a and 12b are adjacent to one another on an elongate and generally planar wand body 14, which in this case has a rectangular shape. Specifically, pads 12a and 12b, which may be formed of any suitable absorbent material as would be recognized by one skilled in the art, are in this case located on the bottom of wand body 14 at one end. Generally, the wand body 14 will be formed a flexible plastic material that facilitates the sample collection process.

The wand body 14 is perforated down the middle (as indicated at 16) between the two pads 12a and 12b, or is otherwise rendered capable of being separated into two halves 14a and 14b. In other words, after sufficient bodily fluid (e.g., saliva) is collected, the collection wand can be easily broken into two halves 10a and 10b (FIG. 6) with each half having one collection pad. After collection, the wand is split at the perforation line so as to supply two identical samples. Two containers containing storage buffer may then be used to preserve the analytes of interest during transport and storage. In lieu of a line of perforations, alternative embodiments are contemplated in which a cut provides a lesser thickness where the body portions are separated. Alternatively, it may be possible to produce halves that are initially separated but are joined with a light adhesive so they can be easily separated again.

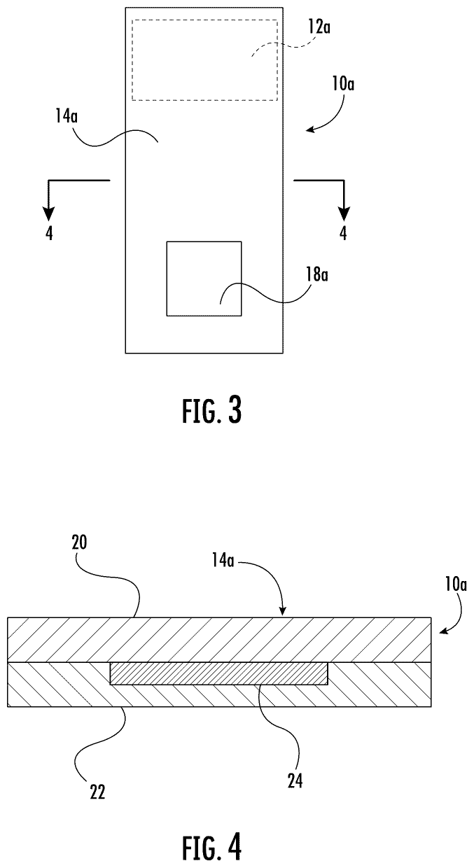

Referring now to FIGS. 3-5, certain additional details regarding the construction of wand 10 will be explained. (Each of these figures shows wand half 10a, although it will be appreciated that wand half 10b is essentially identical.) Each of the wand halves has a respective collection window 18a and 18b that indicates whether a sufficient quantity of sample has been collected. Specifically, the windows 18a and 18b are located at the opposite end from the associated collection pad 12a and 12b on the top of the wand body 18. The user sees a visual indication, such as a change in color or other indicia, when the appropriate sample has been collected.

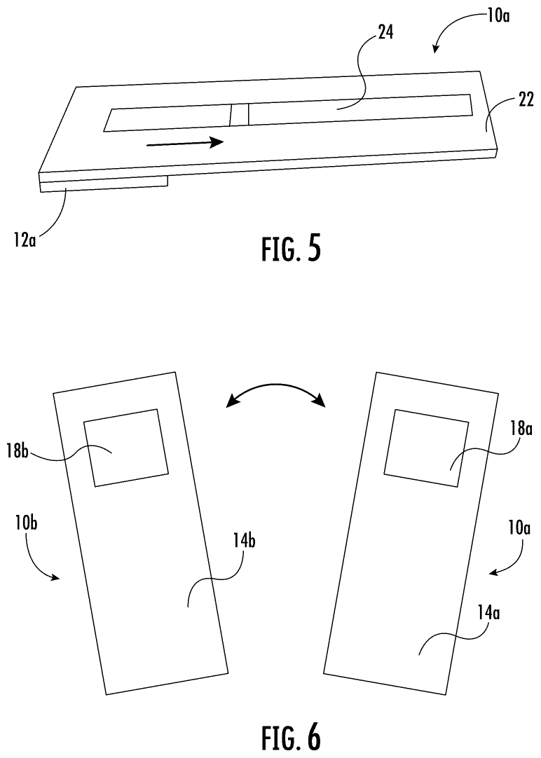

As can be seen in FIG. 4, the wand body 14 is constructed in this case as a multilayer structure having a top layer 20 and bottom layer 22. Referring now also to FIG. 5 (in which top layer 20 is not present), bottom layer 22 carries a suitable wicking element 24 in fluid communication with the associated collection pad 12a or 12b. In this case, the wicking element 24 is located in an elongate channel defined in bottom layer 22. Wicking element 24, such as a strip of suitable paper, extends to a location under the associated window 18a or 18b. (Typically, the window may simply be an aperture in top layer 20 but in some cases it may be desirable to provide a transparent pane in the aperture.) A portion of the bodily fluid collected at the collection pad 12a or 12b will pass to the corresponding wicking element 24 (such as via one or more interconnecting holes extending through bottom layer 22). Some of the sample portion will migrate along the wicking element, eventually reaching a location under the associated window 18a or 18b. The wicking element may, for example, contain indicator die that changes color in the presence of the bodily fluid.

During collection, the wand 10 remains intact as a single piece while the user is instructed to move the pads along their gumline, underneath and on top of their tongue, and between their cheeks. Complete collection is indicated by the presence of dye that is visually observed at the volume sufficiency indicator windows 18a and 18b.

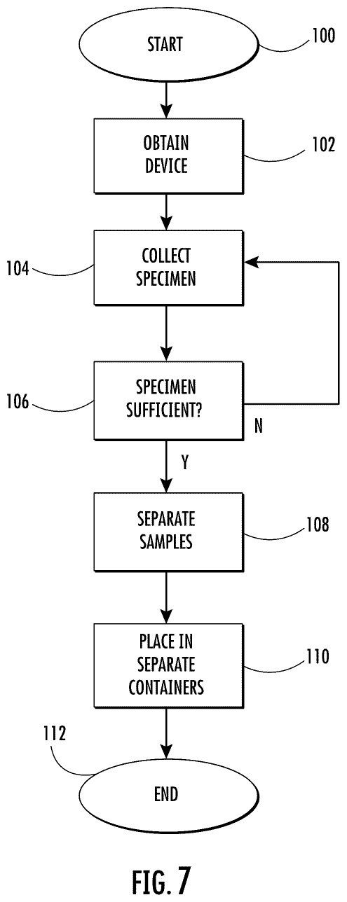

FIG. 7 illustrates an exemplary method in accordance with an aspect of the present invention. The process begins at 100. A suitable device as described above is then obtained, as indicated at 102. The user is instructed how to collect the sample. Once the sample is collected, as indicated at 104, it is determined whether the sample is sufficient, as indicated at 106. If not, collection continues.

If the collected sample is sufficient, the wand is separated into multiple portions, as indicated at 108. The respective wand portions are then placed into separated containers for storage, as indicated at 110. The process then ends, as indicated at 112.

While one or more preferred embodiments of the invention are described above, it should be appreciated by those skilled in the art that various modifications and variations can be made in the present invention without departing from the scope and spirit thereof.

What is claimed is:

1. A bodily fluid sample device comprising:
a wand body having elongate configuration;
the wand body has a first body portion and a second body portion, the wand body being initially unitary but configured so that the first body portion and second body portion can be separated from one another; and
a first collection pad located on the first body portion and a second collection pad located on the second body portion.

2. A bodily fluid collection device as set forth in claim 1, wherein the first collection pad and the second collection pad are adjacent to one another near a first end of the wand body.

3. A bodily fluid collection device as set forth in claim 2, wherein each of the first body portion and the second body portion has a respective first wicking element and a second wicking element in fluid communication with the respective first collection pad and the second collection pad, the wicking elements providing a visual indication that a sample is present.

4. A bodily fluid collection device as set forth in claim 3, wherein the first wicking element and the second wicking element each have an elongate configuration.

5. A bodily fluid collection device as set forth in claim 4, wherein the first wicking element and the second wicking element comprises paper having dye that changes color when the sample is present.

6. A bodily fluid collection device as set forth in claim 3, wherein each of the first and second wicking elements extends under a respective first and second indicator window, the indicator windows being located near a second end of the wand body opposite the first end.

7. A bodily fluid collection device as set forth in claim 3, wherein the wand body has a multilayer configuration with the first and second wicking elements being sandwiched between two layers.

8. A bodily fluid collection device as set forth in claim 1, wherein the wand body defines a line of perforations at which the first body portion and the second body portion can be separated from each other.

9. A bodily fluid sample device comprising:
a wand body having an elongate configuration;
the wand body having a first body portion and a second body portion, the wand body being initially unitary but having a separation line so that the first body portion and second body portion can be separated from one another;
a first collection pad located on the first body portion and a second collection pad located on the second body portion, the first collection pad and the second collection pad being adjacent to one another near a first end of the wand body;
each of the first body portion and the second body portion having a respective first wicking element and a second wicking element in fluid communication with the respective first collection pad and the second collection pad, the wicking elements providing a visual indication that a sample is present at a respective first and second indicator window.

10. A bodily fluid collection device as set forth in claim 9, wherein the first wicking element and the second wicking element each have an elongate configuration.

11. A bodily fluid collection device as set forth in claim 10, wherein the first wicking element and the second wicking element comprises paper treated with dye that changes color when the sample is present.

12. A bodily fluid collection device as set forth in claim 9, wherein each of the first and second wicking elements extends under a respective one of the first and second indicator windows, the indicator windows being located near a second end of the wand body opposite the first end.

13. A bodily fluid collection device as set forth in claim 9, wherein the wand body has a multilayer configuration with the first and second wicking elements being sandwiched between two layers.

14. A bodily fluid collection device as set forth in claim 9, wherein the separation line of formed by a line of perforations at which the first body portion and the second body portion can be separated from each other.

15. A method of collecting a saliva sample from a user, the method comprising steps of:
providing a bodily fluid collection device having an elongate wand body with a first body portion and a second body portion having respective collection pads, the wand body being configured so that the first body portion and second body portion can be separated from one another;
collecting a sample at the collection pads;
determining if the sample is sufficient;
if the sample is sufficient, separating the first body portion from the second body portion; and
separately storing the first body portion and the second body portion.
